(19) United States
(12) Patent Application Publication
(10) Pub. No.: US 2010/0303735 A1
(43) Pub. Date: Dec. 2, 2010

(54) METHOD AND ARRANGEMENT FOR FORMING VARIABLE COLOR PHARMACEUTICAL PRODUCTS

(75) Inventors: Steven L. Yoder, Skippack, PA (US); Frederick R. Kettinger, Norristown, PA (US); Marc Dolbier, Ottsville, PA (US)

Correspondence Address: LUCAS & MERCANTI, LLP, 475 PARK AVENUE SOUTH, 15TH FLOOR, NEW YORK, NY, 10016, US

(73) Assignee: BPSI HOLDINGS, LLC., Wilmington, DE (US)

(21) Appl. No.: 12/682,872

(22) PCT Filed: Aug. 14, 2008
(86) PCT No.: PCT/US08/73191

§ 371 Date: Aug. 18, 2010

Related U.S. Application Data
(60) Provisional application No. 60/956,579, filed on Aug. 17, 2007.

(51) Int. Cl. A61K 9/44; C08J 7/18; C23C 16/52; B29C 35/08

(52) U.S. Cl. 424/10.2; 427/554; 427/8; 264/400

(57) ABSTRACT

Methods and arrangements for forming a color gradient on a pharmaceutical dosage forms such as tablets are disclosed. In preferred aspects of the invention, a coating layer is applied to a tablet core so that the coating layer is different from the color of the tablet core. The coating layer is etched by one or more lasers to remove selected portions thereof and reveal the core. Removal of selected portions of the coating layer, while other portions remain unetched by the laser(s), causes a variation in the color of the coating layer. The color of the tablet core may be visible at locations at which portions of the coating layer have been entirely removed while the color of the coating layer, i.e., the color of any of the color coatings thereof, is visible at locations at which portions of the coating layer have been only partially removed or not removed at all.

### CROSS REFERENCE TO RELATED APPLICATIONS

This application claims the benefit of priority from U.S. provisional application No. 60/956,579 filed Aug. 17, 2007, the contents of which are incorporated herein by reference.

### FIELD OF THE INVENTION

The present invention relates generally to methods and arrangements for forming variable color pharmaceutical products, e.g., oral dosage forms such as tablets, caplets, or capsules, which have multiple colors, shades, tones and/or hues thereon.

The present invention relates more specifically to methods and arrangements for forming graphic symbols, alpha-numeric text or other characters or indicia on tablets and other pharmaceutical products.

### BACKGROUND OF THE INVENTION

Prior art related to color variations in pharmaceutical tablets includes U.S. Patent Application Publication No. 2005/0152971 (Rinker et al.). Rinker et al. relates to an improved gelatinous coated dosage form having two end regions in which a gelatinous material coating is applied over a subcoating, and an exposed circumferential band, identified as a gap by the applicants, in which the subcoating is exposed. In the manufacturing process, an intermediate dosage form is subjected to a mechanical or laser drilling process, e.g., using a transversely excited atmosphere (TEA) laser. The laser is directed through a mask to the circumferential band and/or to one or both of the end regions on the intermediate dosage form to ablate the subcoating and/or gelatinous coating(s) and form openings in a pattern determined by the mask. The core material, underlying the subcoating, is revealed through the openings. The subcoating and/or gelatinous coatings may have a color different than the color of the core material to thereby provide the dosage form with multiple colors.

International Publication No. WO 91/01884 (The Upjohn Company) describes a two-step process for branding tablets wherein first a graphic symbol or geometric shape is printed on the tablet surface using traditional offset printing technology and then a laser marking system is used to remove selected portions of the offset printed ink to expose a coating on the tablet surface beneath the printed ink. The removed portions provide graphic symbols or alpha-numeric text within the ink-printed area of the tablets. To ensure removal of the selected portions of the ink, the laser is controlled to penetrate through the ink into the tablet surface and thus remove a portion of the underlying coating and/or surface material of the tablets.

### OBJECTS AND SUMMARY OF THE INVENTION

A general object of the present invention is to provide a method and arrangement for forming variable color pharmaceutical products.

Further objects of the present invention include providing methods and arrangements for forming film coated compressed tablets, and other oral solid dosage forms such as capsules, having laser etched regions thereon and/or color-variable graphic symbols and/or alphanumeric text thereon.

A still further object is provide oral solid dosage forms, i.e. tablets, or other compressed dosage forms, or capsules, having at least one laser etched region as defined herein thereon.

For purposes of description rather than limitation, the invention is usually described herein with reference to a preferred embodiment which includes compressed tablets. The invention however is not limited to compressed tablets and where reference is made to compressed tablets or tablet cores, it is to be understood that all other pharmaceutically acceptable oral solid dosage forms, including capsules are intended.

In order to achieve at least one of these objects and possibly others, methods for forming pharmaceutical tablets or oral dosage forms having a laser etched region or color gradient thereon are provided. The methods include:

applying at least one color coating onto an oral solid dosage core, preferably a tablet core, to form a coating layer, the at least one color coating having a color different than the color of the oral solid dosage or tablet core; and

laser etching a portion of the coating layer to remove a selected portion thereof or render the color of the oral solid dosage or tablet core visible at the location at which the selected portion of the coating layer has been removed.

Removal of selected portions of the coating layer, while other portions remain unetched by the laser, causes a variation in the coating layer; it is present at the unetched portions and either partially or entirely removed at etched portions. Accordingly, the color of the oral solid dosage or tablet core is visible at locations at which portions of the coating layer have been entirely removed while the color of an underlying coating is visible at locations at which portions of the coating layer have been only partially removed.

Removal of the coating layer, either a partial thickness of the entire thickness, constitutes a reverse pixelation since individual portions or pixels of the coating layer are removed by the laser. The location, density and size of these pixels are adjusted via control of the laser to enable formation of different color variations, including for example, a uniform color variation in a linear direction along the oral solid dosage form or tablet (i.e., a fading color gradient).

Multiple lasers, or a single laser using a multi-pass arrangement, may be used to remove different thickness of the coating layer. This would be useful when the coating layer has multiple color coatings in which case, the lasers could remove only the uppermost coating from some portions of the coating layer, and all of the coatings from other portions to thereby increase the color variation.

In some preferred aspects of the invention, methods are provided wherein a plurality of coatings are applied onto an oral solid dosage or a tablet core. As an example of this embodiment, successive layers of film coatings, e.g. 3 or more, each different in color are placed around a tablet core having yet another color, so that the color of each layer or the core in contact therewith differs are made. Thereafter, the method includes
a) laser etching a first design such as a circle or other desired shape onto the tablet core by removing contiguous portions of each of the layers to expose the core in the desired design; next, the method continues with
b) laser etching a second design, such as a drug name, manufacturer's name, a slogan or a logo, onto the film coating layer in contact with the tablet core. The second design is separate from the first design and is formed by removing contiguous portions of each of the second and the third coating layers.

In further aspects of this embodiment, the method of can include
c) laser etching a third design onto the second film coating layer of the pharmaceutical tablet. The laser etching is carried out so that the third design is separate from each of the first and the second designs, by removing a portion of the third coating layer.

In yet another embodiment, there are provided various pharmaceutical dosage forms such as pharmaceutical tablets (preferred) prepared by any of the processes described herein.

The pharmaceutical tablets of the invention have a laser etched region thereon prepared by forming a coating layer having a first color onto a tablet core having a second color, with the first color being different than the second color. Once coated, laser etching is carried out on a portion of the coating layer to remove a portion thereof and to reveal a selected portion thereon. This reveals the second color of the tablet core where the selected portion of the coating layer has been removed.

In yet a still further aspect of the invention, the method includes orienting the laser at an angle which is not perpendicular to a horizontal axis or surface of each tablet. This creates a holographic effect, i.e., the etched pattern changes as the tablet is turned relative to the viewer. Multiple etchings at different angles can be used to create a holographic animated effect, i.e., a pattern can be formed which changes colors as the tablet is turned relative to the viewer.

In another embodiment, to enhance the possible color gradients or variation, one or more of the color coatings is/are applied to only a portion of the tablet or the immediately underlying coating. This therefore increases the number of colors, shades or hues visible on the coated tablet and thus the color variability of a pattern to be etched onto such a coated tablet.

An arrangement for forming pharmaceutical tablets with a color gradient in accordance with the invention includes at least one coating system arranged to apply at least one color coating onto tablet cores to thereby form a coating layer, the at least one color coating having a color different than the color of the tablet cores, and a laser etching system arranged to remove selected portions of the coating layer. The laser etching system causes a variation in the coating layer so that, for a coated tablet with a single coating in the coating layer, the color of the tablet cores is visible at locations at which the selected portions of the coating layer have been removed.

When the coating layer includes multiple color coatings, the laser etching system is effective to remove different portions of each color coating so that for some portions, the color of the tablet core is visible while for others, the color of a first color coating is visible and for others, the color of the second or additional color coating(s) is visible. Within this embodiment, a preferred aspect of the invention can include using a laser to completely remove a significant portion or side of a coating layer found on a tablet core. Such use of the laser results in the removal of the layer without pixelation. Thus, contiguous portions of the coating layer, such as one or more bands or stripes of the coating layer, may be removed instead of individual, isolated spots as are removed when the coating layer is pixelated.

In an alternative embodiment of the invention, a tablet core is enrobed with a pharmaceutically acceptable capsule (e.g. gelatin) designed to include overlapping portions. The upper and lower capsule shell portions of the capsule can be the same color, or, as in some preferred aspects of the invention, different colors. The laser etching techniques described herein are carried out on the upper capsule shell portion which overlaps the lower shell portion and thus reveals the preferably contrasting color of the lower shell portion. In alternative embodiments, both the overlapping portion of the upper capsule shell and the lower shell portion are laser etched to reveal the underlying color of the optionally color coated tablet core and achieve one or more of the visual effects described herein. It will be appreciated that when gelatin capsules are used to encapsulate powdered formulations, the laser etching is done only to the overlapping portion of upper shell.

The coating system and laser etching system can be arranged to perform the steps of the method described above.

### BRIEF DESCRIPTION OF THE DRAWINGS

The invention, together with further objects and advantages thereof, may best be understood by reference to the following description taken in conjunction with the accompanying drawings, wherein like reference numerals identify like elements, and wherein:

FIG. 1 is a schematic of a method for forming a coated tablet in accordance with the invention.

FIG. 2 is a photograph of tablets showing various levels of laser etching.

FIG. 3 is a photograph of various tablets illustrating various changes in hue achieved by the invention and Example 2.

FIG. 4 is a schematic of a system for forming coated tablets in accordance with the invention applying a continuous manufacturing operation.

FIG. 5 is a photograph of a tablet having a laser etched image thereon, prepared according to Example 1.

FIG. 6 is a photograph of a tablet having a laser etched image thereon, prepared according to Example 3.

FIG. 7a is an acetaminophen capsule described in Example 4 prior to undergoing laser etching.

FIG. 7b is an acetaminophen capsule described in Example 4 illustrating laser etching of an outer shell portion.

### DETAILED DESCRIPTION OF THE INVENTION

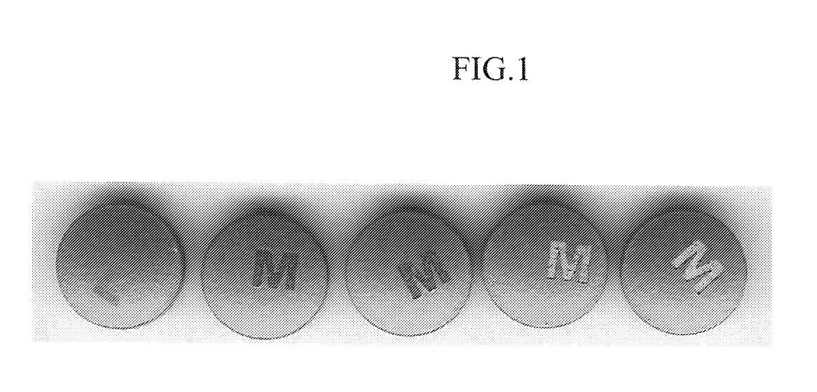

Referring to the accompanying drawings wherein the same reference numerals refer to the same or similar elements, FIG. 1 is a flow chart of a method for forming a color or film coated tablet in accordance with one aspect of the invention.

The first step 10 in the method corresponding to this embodiment is to form the tablet cores, which may be accomplished using any tablet manufacturing process known to those skilled in the art for making compressed tablets. For purposes of the present invention, it will be understood that the term “tablet core” refers to the compressed tablet prior to undergoing the film coating(s) and laser etching desired. The shape and size of tablet cores that can be used in the invention are essentially unlimited. Obviously, the tablet cores would include the active ingredient(s) of the tablet, excipients and other necessary ingredients to deliver the active ingredient(s).

The second step 12 is to spray or otherwise apply one or more coatings to the tablet cores. Where more than one coat is applied, the first coating of the core is referred to as the subcoat, with successive layers applied thereto ultimately finishing with a top coat. The manner in which color coatings are applied directly onto the tablet cores, or directly onto a previously applied coating, may be any tablet coating process known to those skilled in the art. Moreover, known tablet coating processes whereby only portions of the tablet are coated, such as dipping, may also be used.

Exemplifying technologies for coating tablets include without limitation pan coating, spray coating, enrobing, gel dipping, sugar-coating and electrostatic coating or coloring technologies. The invention is not limited to any specific technology for coating the tablets, whether these listed technologies or any other technologies currently known in the art or any subsequently developed technologies.

For this step, the color of the coating or coatings is preferably selected to enable a color contrast between the color of the tablet core and/or the color of the coating or coatings. Thus, for example, if a single coating is being applied to the tablet core, the color of the single coating would be different than the color of the tablet core and preferably a color which enables a high contrast to the color of the tablet core.

In those aspects of the invention where two coatings are applied onto the tablet core, to maximize the potential color contrast of the coated tablets after further processing described below, each coating would preferably have a color different than the color of the tablet core and different from the color of the other coating. Preferably, the color of each coating is selected to enable a high contrast with the color of the tablet core and the color of the other coating. Instead of or in addition to maximizing the potential color contrast by appropriate selection of the color of coatings, it possible to maximize the distinctiveness of the coated tables by forming a holographic image. This is achieved in the subsequent laser etching stage described below.

Similarly, when three or more coatings are applied onto the tablet core, to maximize the potential color contrast of the coated tablets after further processing described below, each coating preferably has a color different than the color of the tablet core and different from the color of the other coatings. Preferably, the color of each coating is selected to enable a high contrast with the color of the tablet core and the color of the other coatings.

Each coating may be applied to the entire outer surface of the tablet, which is either the outer surface of the tablet core if the coating is the first coating or the outer surface of a previously applied coating if the coating is a subsequent coating.

Alternatively, each coating can be applied only to a portion of the outer surface of the tablet. In this case, the potential color variations of the coated tablet after subjected to further processing described below, can be significantly increased. For example, if a green coating is first applied onto one-half of a white tablet core, then a yellow coating is applied onto the other, uncoated half of the tablet core and a portion of the previously coated half, and then a red coating is applied onto a portion of the only green-coated portion, only a portion of the yellow-coated portion and only a portion of the green and yellow-coated portion, there would be a total of five different colors on the outer surface of the tablet. Specifically, there would be a green portion, a yellow portion, a red over green portion, a red over yellow portion and a red over green and yellow portion. The presence of these multiple and different colored portions will allow for a significant increase in the color variability of the tablet after laser etching, described below. It will be understood from the foregoing that the invention is not limited to the number of color layers applied to the tablet core.

Each coating can be considered to form a discrete sublayer on the tablet. The combination of the coatings may be considered a coating layer.

The types of coatings which may be applied to the tablet cores can vary widely according to the needs of the artisan and the type of coating effects desired by the artisan. In most aspects of the invention, commercially available film coatings for tablets such as those available from Colorcon, including without limitation, Opadry®, Opadry® II, Opadry®fx, Opadry® AMB and Opaglos® or others known to those of ordinary skill can be used. In some preferred embodiments, film coatings containing PVA such as the aforementioned Opadry II products are used in coating the tablet cores prior to laser etching.

The coating or coatings may be applied in a batch, semi-batch or continuous operation, described below. If multiple coatings are applied, then they could be sequentially applied in any of the batch, semi-batch or continuous operation methods. The particular coating sequence could be determined to optimize the ability to provide a high color contrast between the coatings and/or the tablet core without undue experimentation.

In one embodiment, the quantity or thickness of the coating layer applied to the tablet is a function of the weight of the tablet. The film coating can be applied as part of a pan coating or spray coating process commonly used to coat such articles. The amount of coating applied will depend upon several factors, including the substrate to be coated, the amount and color of the pigment included in the film coating solution or suspension, the apparatus employed to apply the coating, etc. In most aspects of the invention, however the cores will be coated to a theoretical weight gain of from about 0.25 to about 5.0%. Preferably, the theoretical weight gain is from about 0.5 to about 4.0% and more preferably, the theoretical weight gain is from about 1.0 to about 3.0% by weight of said substrate. For example, in one preferred embodiment, the coating layer may have a weight of about 1% to about 3% of the total weight of the tablet while the top coat, i.e., the last coating, has a weight of about 3% of the total weight of the tablet. If multiple coating layers are used, it is preferred that the total amount of weight gain attributable to the film coatings be within the range provided above.

Once the tablet cores are coated with one or more coatings, the next step in one preferred aspect of the invention, 14 (with respect to the flow diagram of FIG. 1) is to laser etch into the color coating or coatings a graphic symbol, alphanumeric text, other types or forms of symbols or lettering, or combinations thereof. The form being laser etched thus includes, but is not limited to, words and other indicia, such as designs, logos, figures, serialized numbers, trademarks, two or three-dimensional bar codes and registration marks.

There are several acceptable techniques to perform laser etching. One technique is to reverse pixelate the color coating which generally entails the removal of individual, isolated spots of the color coating, each spot being considered a pixel. As a result of applying such a reverse pixelation technique, the color coating will have any number of isolated regions in which the color coating has been removed and the subcoating is visible surrounded by the coating which has been laser etched. The laser etching can be performed so that the laser is angled substantially perpendicular to the color coating being etched or alternatively, at a non-perpendicular angle to the color coating. In the latter case, the reverse pixelation forms a holographic image which is visible upon rotation of the tablet. Additional details about formation of holographic images is set forth below.

Another technique, instead of reverse pixelation, is to laser etch the coating layer in order to remove contiguous regions of the color coating. In this case, a band of the coating layer may be removed so that the subcoating is visible in this band surrounded on either side by a non-etched portion in which the coating layer is visible. Multiple bands, which extend circumferentially around the tablet, or stripes, which extend only partially around the circumference of the tablet, may be formed. In addition, other contiguous regions having geometrical or non-geometrical forms may also be laser etched, for example, one or more circles.

Although there are known lasers which can be used for laser etching, one particular laser that has been found to be useful in a method in accordance with the invention is a YAG laser designated a Cobalt 1000 model sold by ICN. Such a laser is operative to vaporize the surface that is being etched, as opposed to burning the surface as would typically occur when other lasers are used. Thus, using the YAG laser, there is no apparent chemical change, charring or burning of the coating layer or the tablet core. Other lasers capable of achieving the desired result on the tablets are also contemplated as being part of the invention.

As described above, a laser may be used to etch into the coating layer in order to reverse pixelate the coating layer since specific portions, or pixels, of the coating layer are being removed. The density and/or size of the pixels being removed from the coating layer is adjustable and may be determined, for example, by the settings of the operating parameters of the laser. The density of the pixels is the number of holes (individual removed portions of the color coating layer) per unit of area from which a color coating is removed by the laser during the etching step. The laser may thus be considered to use a mask which prevents the application of the laser to certain areas while allowing the application of the laser to other areas and thus causing removal of portions of the color coating to which the laser is applied.

The operating parameters of the laser can also be controlled to enable formation of different color variations. That is, the laser can be controlled to selectively remove different thicknesses of a color coating, or the color coating in its entirety, to create a different visual appearance of the etched portion of the tablet relative to unetched portions of the tablet and relative to differently etched portions of the tablet. The controlled parameters of the laser include, but are not limited to, its power, frequency, hatch and speed. For example, lasers between 3 and 50 watts of power, in a range of wavelengths between about 1 and 10 microns have been successfully used in carrying out the invention, with the smaller wavelengths within the range providing preferable results on film coated tablets. The YAG and Yb Fiber lasers fall at the ends of the spectrum. CO2 lasers have wavelengths of about 10.64 microns. YAG and Yb fiber lasers between about 0.8 and about 1.064 microns are also useful. For purposes of illustration, the use of a lab scale YAG laser with a wavelength of 1.064 microns yielded very good etching results of film coated tablets. The power settings for the lasers are preferably uniformly low—in a range from 2%-10% of maximum. The hatch pattern, when used, varied from 0.1 to 0.8 (no units specified). This setting only applies to designs that require removal of a large amount of material. Alphanumeric characters generally do not have a hatch applied. The speed of the beam's motion is usually quite fast, in the range of 70%-95% of maximum. Adjustment of the power of the laser, in particular, results in a variation in the thickness of the color coating(s) which is removed.

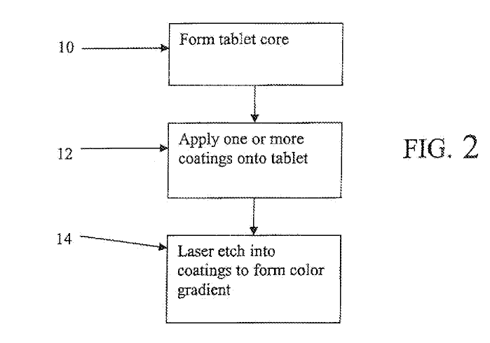

FIG. 2 shows the effect of varied power levels on etching. In this illustration, speed was kept constant (50) while the power level was varied. This resulted in the incremental increase in the amount of coating removed. Each tablet was etched separately. The power levels represented above were 5%, 5.5%, 6.0%, 7.0%, and 7.5%. The film thicknesses were top to bottom 1%, 3%, 3%, 3% by weight corresponding to a thickness of 10 microns, 30 microns, 30 microns, and 30 microns, (FX, Red, Green, Yellow).

By adjusting one or more of the operating parameters of laser during formation of a graphic symbol or alphanumeric text on a single tablet, it becomes possible to create a color gradient on that tablet. That is, in one preferred aspect of the invention, the color coating can be provided with a thickness which varies from one portion of the tablet or graphic symbol found thereon or alphanumeric text to other portions and the variations in the thickness of the color coating result in a multi-color or colorshifting appearance of the tablet since the appreciation of the color of a coating depends on its thickness. A thinner coating of the same color will thus appear lighter than a thicker coating of the same color. More generally, a thinner coating of the same color will create a different color effect in combination with the underlying or overlying color than the thicker coating of the same color.

The colorshifting aspect arises from the presence of one color at one area of the tablet and another color or another hue or shade of the same color at another area of the tablet. When viewing the tablet, there is a visual variation from one area to the other. Appropriate selection of the colors and mask for the laser can provide striking color gradients which can be used to enhance the branding of the tablets, described more fully below along with other uses of the formation of a color gradient in accordance with the invention.

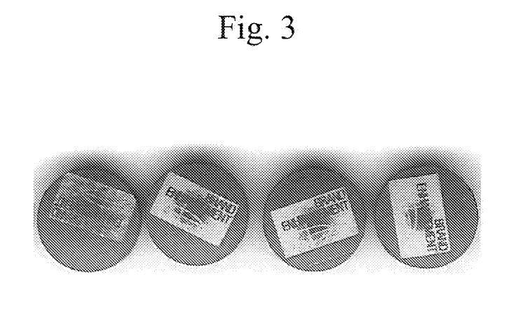

For example, in this aspect of the invention there is provided a “fading” color appearance from a first side of the film coated tablet or graphic symbol or alphanumeric text found thereon to a second side. This is achieved by controlling the laser to etch more of the same color coating at the first side than at the second side, with a gradual decrease in the etching of the color coating from the first side to the second side. In this manner, the amount of the color coating remaining after the laser etching step varies gradually from one side of the graphic symbol or alphanumeric text to the opposite side resulting in a lighter shade or hue of the color of the coating at the first side, a darker shade or hue of the color of the coating at the second side, and transition shades or hues in the middle. See FIG. 3 and Example 2 below wherein the tablets shown were exposed to increasing amounts of energy. Using a gray scale image to map the power level of the laser produced the variation from yellow to green. Where the image was white, the laser did not remove material, where the image was black, it removed material at full power, where it was gray, it removed a fraction of the material.

It is important to appreciate that this particular fading effect is only one type of fading effect that can be generated by control of the laser to remove 25′ different thickness of the same color coating. Other possible fading effects include fading from each side of the film coated tablet or graphic symbol or alphanumeric text thereon to a middle, with either the middle being subject to the largest reduction in the thickness of the color coating or the least thickness reduction.

Control of the laser to remove different thicknesses of the coating layer may require the tablets to pass by the laser two or more times, with the laser being set differently each time. The laser etching process would thus require the tablets to be etched a first time, resetting of the laser and the etching of the tablets a second time. Additional resetting of the laser and etching of the tables is also possible, depending on the particular color gradient sought on the tablets.

Alternatively, it is possible to obtain a multi-color appearance on tablets by using multiple lasers. When multiple lasers are used, the etching being performed by each laser would be different so that a different form is etched into the coating layer by each laser and the combination of the different etched forms creates the multi-color appearance. The different etching may involve use of a different mask, different settings and/or different relative position between the tablets and the laser (discussed below).

When a single laser is used and the tablets are passed by the laser two or more times, each pass being considered a loop, at least the etching mask of the laser for each loop would be different so that a different form is etched into the coating layer in each loop. The combination of the different etched forms creates the multi-color appearance. For example, it is possible to partition the graphic symbol or alphanumeric text into two parts, with one mask being formed for each part. The operating parameters of the laser are set for the first loop when one mask is applied to cause the laser to remove a specific thickness of the color coating in accordance with the mask, and thereby create one part of the graphic symbol or alphanumeric text. The operating parameters of the laser are then changed for the second loop when the other mask is applied to cause the laser to remove a different thickness of the color coating in accordance with this mask, and thereby create the other part of the graphic symbol or alphanumeric text. When viewed in its entirety, the graphic symbol or alphanumeric text has one part of one thickness and another part of a different thickness, to thereby create the multi-color appearance.

The laser etching step can also be effective to create a pearlescent logo, graphic symbol or alphanumeric text. In this case, a pearlescent subcoat is applied onto the tablet core and one or more coatings are applied onto the pearlescent subcoat. The laser etches the coating(s) to expose the pearlescent subcoat, in a particular pattern as determined by the mask used with the laser, with the result that a pearlescent logo, graphic symbol or alphanumeric text is created. The degree to which the pearlescent logo, graphic symbol or alphanumeric text can be seen varies depending on, for example, the color or colors of the coating(s).

In the laser etching process, there is the potential for adverse interaction between the laser and the color or composition of certain coatings. Discoloration of the tablet is not desirable and therefore, through routine experimentation, it can be determined which operating parameters of the laser do not cause discoloration of specific color coatings and thus which combinations of laser operating parameters and color coatings can be effectively used in the invention.

The manner in which the four steps set forth in FIG. 1 and described above are implemented may be any known tablet production process. The tablet production processes which provide multi-color tablets include, but are not limited, to batch processes, semi-batch processes and continuous operations.

In a batch process, a single coating system would be used and feed with two or more different color coatings. The coating system would spray or otherwise apply the coatings onto tablet cores to create a coating layer of different coatings.

A laser etching system would follow the single coating system and reverse pixelate the tablets by removing specific portions of the coating layer (as described above). An inspection system and packaging system would follow the laser etching system.

In a semi-batch process, two or more batch coating systems are arranged in a series, each feed with a single color coating. The tablets would be directed in succession past each coating system which would spray or otherwise apply its coating onto the tablet. After the last coating system, the tablets would have a coating layer of different coatings. A laser etching system would follow the last coating system and reverse pixelate the tablets by removing specific portions of the coating layer. An inspection system and packaging system would follow the laser etching system.

In one embodiment of the invention, tablets are coated using an electrostatic coating technique to form a coating layer and then the coating layer is laser etched or reverse pixilated to create a pattern in the form of a graphic symbol or alphanumeric text, or combination, with a color gradient. In this case, reverse pixelating the coating layer provides a significant advantage to a case where the graphic symbol or alphanumeric text is electrostatically deposited onto the tablet surface. Electrostatic deposition of the graphic symbol or alphanumeric text does not result in a clearly focused graphic symbol or alphanumeric text when applied onto a curved surface of the tablet. By contrast though, reverse pixelation results in a clear, focused graphic symbol or alphanumeric text on the tablet, regardless of the shape of the tablet.

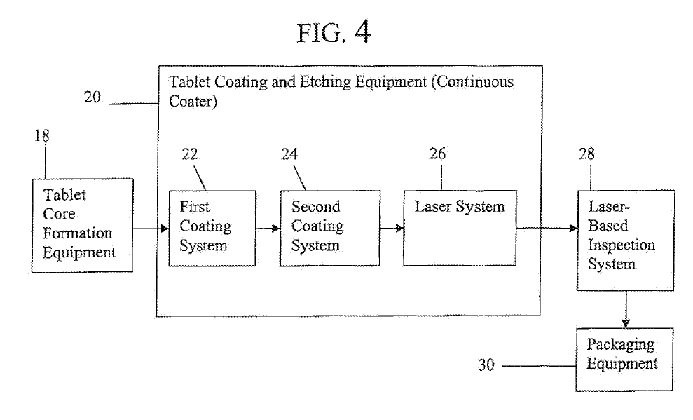

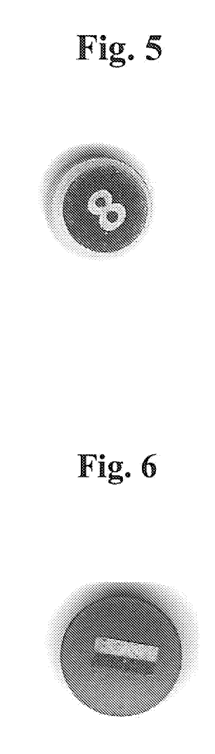

FIG. 4 shows a tablet manufacturing system applying a multi-color continuous tablet coating operation. Initially, tablet cores are formed by a machine 18 known to those skilled in the art. The tablet cores may be formed by machine 18 at the site of the coating and etching equipment 20 or distant therefrom and conveyed to a storage device proximate the coating and etching equipment 20.

The tablet cores enter the coating and etching equipment 20 and pass by or through a first coating system 22 which sprays or otherwise applies a first color coating onto a portion of or all of the surface of each tablet core. Once coated with the first color coating, the coated tablets proceed to a second color coating system 24 which sprays or otherwise applies a second color coating onto a portion or all of the first color coating on the tablet cores.

The coated tablets then proceed to the laser etching system 26 wherein one or more lasers etch a pattern onto the coating layer. As described above, the laser etching system may include a YAG laser which removes individual portions of the coating layer without affecting other portions. To effect the laser etching, the tablets are be placed into an etch chamber of the YAG laser, and the graphic symbol or other design to be etched is prepared using software of a control unit for the laser. The software sends instructions to the laser to set its operating parameters, e.g., frequency, speed and power, and etch the tablet in accordance with a mask, or masks if multiple loops are required, which will provide the graphic symbol or other design. The control unit software is preferably designed to receive an image, such as in the form of a commercially available format such as an Adobe Illustrator or .dxf file, and enable conversion of the image into appropriate control instructions for the laser.

For smaller scale runs, the tablets were placed on a carbon block with support to allow the laser to expose each tablet from the same angle. At higher rates, each tablet can be placed in a pre-formed, contoured pocket with or without suction from below to hold it in place and the laser will track with the tablet as it etches.

Once the pattern is etched onto the coating layer, the tablets proceed to an inspection system 28 and packaging equipment 30.

The inspection system 28 may be any known tablet inspection system which inspects tablets to identify irregularities in the formation of the tablets, and may also be designed to determine irregularities in the coating processes and the laser etching process. To this end, one possible inspection system is a laser-based inspection system which uses spatial color analysis technology to verify the color of the tablets. Additionally, the count and shape of tablets, the position of a graphic symbol or alphanumeric text, and presence of a graphic symbol or alphanumeric text can be verified.

An exemplifying inspection system 30 would be an existing laser-based inspection system which uses a plurality of color cameras, e.g., two to six, and is designed to identify color errors, shape effects, missing doses, etc. Such a laser-based inspection system could thus be used in the invention to determine irregularities in the color gradients of the graphic symbols or alphanumeric text formed on the tablets in a method in accordance with the invention.

Using a method in accordance with the invention, a multi-color coating layer or single color coating layer which contrasts with the color of the tablet core can be imaged by laser etching to provide an optically active color image similar to a hologram or holographic image. This holographic effect can be created by reverse pixelating or etching an image or logo, such as an eagle or imprint code, by using the laser etching process at one or more angles which are not perpendicular to the horizontal axis or surface of the tablet. When the coating layer is multi-color and laser etched, varying the angle of the laser relative to the tablet being coated will result in different visual effects depending on the angle at which the tablet is viewed after etching.

In this case then, it is the position of the laser relative to the tablets, i.e., the angle the laser beams forms with the surface of the tablet being coated, which can be varied to create not only a color gradient, but also a holographic effect.

For example, a tablet that has a blue coating underneath a red coating underneath a yellow top coat can be laser etched or reverse pixelated such that when observing the tablet surface, the laser is aimed into the surface of the tablet at an angle (such as 45 degrees from the left) and controlled to etch down to the blue coating. When viewing the tablet from front the left at a 45 degree angle, the image is visible as a blue etch. In a similar manner, when observing the tablet surface, the laser can be also aimed into the surface of the tablet at an angle (such as 45 degrees from the right) and controlled to etch down to the red coating. When viewing the tablet from the right at a 45 degree angle, the image is visible as a red etch. By tilting the tablet from left to right and back to left, the image can be observed to change color alternately from blue to red to blue, all on a yellow tablet.

By changing the colors and number of colored coatings, any desired holographic effect can be obtained.

Furthermore, it is possible to apply a method in accordance with the invention to provide holographic animated tablet images on the surface of a tablet. For example, using in the tablet coated as described immediately above, an image of an eagle in-flight with its wings depressed can be reverse pixelated or etched at a 45 degree angle from the left (when looking at the face of the tablet) down to the blue coating. Likewise, the same eagle in-flight with its wings lifted upward can be reverse pixelated or etched at a 45 degree angle from the right (when looking at the face of the tablet) down to the red coating. By tilting the tablet from left to right and back again, the eagle will be observed to move its wings in flight in a down (or depressed) then lifted (or upward) then down again while the color of the wings will change from blue to red to blue, all on a yellow tablet.

Various combinations of the above concepts can produce multi-color images (resulting from a multi-color coating layer) as well as two-color images (resulting from a single color coating layer on a different color tablet core) on a tablet. Additionally, the images can change upon tilting of the tablet. The images can also be created to move in step fashion across the face of the tablet, or around the periphery of the tablet. In combination with pearlesence coatings (as either a coating or the top coat), the optical activity of the color can be enhanced.

The tablet coating and laser etching method described above can be used in several different ways to provide distinct benefits. Generally, the method is effective to enable tablets to be differentiated from one another by creating on each tablet a distinctive laser etched patterns, whether a graphic symbol, alphanumeric text or combination of the two, or any other type or form of design, some of which are mentioned above. The degree of differentiation varies based on the use of different colors, and the interaction between colors when multiple coatings are used, so that as more colored coatings are used or become available for tablet coating purposes, the degree of differentiation increases.

Accordingly, one particular use of the method in accordance with the invention is for branding tablets in which a company creates a unique or distinctive branding mark on tablets so as to make the tablets readily recognizable as a product of that company, or as a specific product marketed by that company possibly under a tradename or trademark.

Another use is for anti-counterfeiting in which tablets are provided with a particular graphic symbol or alphanumeric text which is difficult if not impossible to duplicate. One reason for this is because the laser etching process can etch a distinctive pattern into the coating layer of tablets, using a mask or the like, to form tablets having uniquely colored graphic symbols and alphanumeric text. So long as the pattern is maintained secret, it would be virtually impossible to reverse engineer the tablet to obtain the laser etching mask.

In this regard, a security code may be laser etched onto each tablet by the manufacturer. Such a security code would be generated to enable it to be read by a suitable, possibly handheld, scanner. Placement of the scanner at a pharmacy or other tablet distribution site, and use of the scanner when dispensing the tablets, would quickly determine whether the tablet is genuine or counterfeit. The security code may be indicative of a combination of data about the tablet, such as its batch number, National Drug Code number, lot number, place of manufacture, expiration date, etc.

Another use is for tablet identification purposes in which a company can create a specific colored graphic symbol or alphanumeric text on tablets to enable such tablets to be identified from among other, for example, similarly shaped tablets.

Yet another use is for tablet authentication in which a company can create a specific colored graphic symbol or alphanumeric text on tablets, the presence of which is indicative of the authenticity of the tablets.

A significant benefit of the tablet coating method in accordance with the invention is that by forming uniquely and variably colored graphic symbols or alphanumeric text on different tablets, medication errors can be reduced if not eliminated. For example, the laser etching can create on the tablet surface, an indication of the weight of the tablets or the concentration of the active ingredient in the tablets, in a high contrast color scheme so that the weight or concentration would be difficult to overlook when giving the tablet to a patient.

In another embodiment of the invention there are provided multicolor layered film coated capsules which can be laser etched as described above. Pharmaceutical capsules can be film coated using pharmaceutically acceptable film coatings in the manner described above with respect to the film coating tablets. The capsules are coated with one or more discrete colored layers of film coatings and the resulting product can be laser etched in the same ways as described above with respect to tablets. Alternatively, multicolor capsule shells, i.e. the upper capsule shell and the lower capsule shell are different colors, can be used. In this embodiment, the upper and lower capsule shells are designed so as to permit sufficient overlap in the central region when assembled in final form. It is estimated that for most aspects of this embodiment, overlaps in the region of about 5 to 20% of the surface area of the final capsule will be sufficient. Using the techniques described herein, the laser etching can reveal a portion of the overlapping upper capsule shell area to reveal the lower portion of the capsule shell and provide a unique appearance. In yet a further alternative embodiment, where the multicolored overlapping capsule shells are used to encapsulate a compressed caplet, the laser etching can be carried out to remove a portion of both the upper and lower capsule shell portions in the overlapped region to reveal the underlying, optionally film coated compressed tablet or caplet. The capsules can have images and/or protective identification such as a graphic symbol or barcode thereon.

In still further aspects of this capsule embodiment, multicolor or single color capsule dosage forms are prepared which are sealed with a capsule band having a contrasting color. The capsule band can be laser etched to provide a distinctive final product.

As can be seen, these embodiments of the invention provide optionally film coated gelatin capsules or capsules made with other suitable materials which can be laser imaged. It is contemplated that any pharmaceutically acceptable capsule material, such as gelatin, can be used in accordance with the present invention.

Examples of tablets formed applying the method in accordance with the invention are as follows:

Power levels, frequency, hatch and Speed for different examples:

| | | | Hatch (in mm | Speed |
|---|---|---|---|---|
| | | | vs. angle from | (max = |
| Color | Power % | Frequency in | horizontal) | 1000) |
| Yellow to Green | 9.460 | 10 | .009 (45 degree) | 800 |
| Red to Yellow | 9.460 | 20.02 | .015 (90 degree) | 736 |
| Red to Yellow | 9.053 | 20.02 | .013 (45 degree) | 755 |
| White to Red | 5.000 | 20.02 | .013 (45 degree) | 800 |
| White to Green | 5.000 | 20.02 | .013 (45 degree) | 750 |
| White to Yellow | 6.000 | 20.02 | .013 (45 degree) | 750 |
| White to Red | 8.500 | 20.02 | .016 (45 degree) | 750 |
| Yellow to Green | 8.500 | 20.02 | .016 (45 degree) | 750 |
| Red to | 5.000 | 20.02 | .013 (45 degree) | 750 |
| Green/Yellow | | | | |
| (2^{nd} loop) | 5.000 | 20.02 | .013 (45 degree) | 750 |
| White to Red/ | 5.000 | 20.02 | .013 (45 degree) | 750 |
| Green | | | | |
| (2^{nd} loop) | 5.000 | 20.02 | .013 (45 degree) | 750 |
| White to | 5.000 | 20.02 | .013 (45 degree) | 750 |
| Red/Yellow | | | | |
| (2^{nd} loop) | 6.00 | 20.02 | .013 (45 degree) | 750 |

| Color | Power | Speed |
|---|---|---|
| Yellow to Green | 9.460 | 800 |
| Red to Yellow | 9.460 | 736 |
| Red to Yellow | 9.053 | 755 |
| White to Red | 5.000 | 800 |
| White to Green | 5.000 | 750 |
| White to Yellow | 6.000 | 750 |
| White to Red | 8.500 | 750 |
| Yellow to Green | 8.500 | 750 |
| Red to Green/Yellow | 5.000 | 750 |

### Example 1

A tablet core was first coated with a green coating, 3% by weight, and then by a yellow coating, 3% by weight. A logo was laser etched into the tablet using a YAG laser set at power of 9.6, frequency of 10, hatch of 0.009 at 45° and speed of 800 to remove selected portions of the yellow coating. The coatings were removed selectively to provide a two-color film coated tablet. See FIG. 3.

### Example 2

A tablet core was first coated with a yellow coating, 3% by weight, then by a green coating, 3% by weight, and then by a red coating, 3% by weight.

A logo was laser etched in a two-loop process. In the first loop, a portion of the logo was laser etched—power 5, frequency 20.02, hatch 0.013 45°, speed 750 to remove selected portions of the red coating which overlie the yellow coating and the yellow and green coatings. In a second loop, a portion of the logo was laser etched—power 5, frequency 20.02, hatch 0.013 45°, speed 750 to remove selected portions of the green coating which overlie the yellow coating. The thickness of the removed portions was varied through operation of the laser, and thereby provide a red to yellow color gradient, a red to yellow/green color gradient and a green to yellow color gradient

Referring now to FIG. 3, the tablets shown were exposed to increasing amounts of energy. Using a gray scale image to map the power level of the laser produced the variation from yellow to green. Where the image was white the laser did not remove material, where the image was black it removed material at full power, where it was gray it removed a fraction of the material.

### Example 3

A tablet core was first coated entirely with a yellow coating, 3% by weight, then entirely with a green coating, 3% by weight, and then entirely with a red coating, 3% by weight.

The coating layer was laser etched in a two-loop or two-stage process. In the first stage, the coating layer was laser etched to remove a single band or stripe of the red coating to thereby expose the immediately underlying green coating. In the second stage, the coating layer was laser etched, at different laser settings (described below) to remove a single band or stripe of the combined red and green coating, at a different portion of the coated tablet from which the single band or stripe of red coating was removed, to thereby expose the immediately underlying yellow coating. The resultant coated tablet, thus has an overall red appearance with a single green band or stripe and a single yellow band or stripe. See FIG. 6.

| | | | | |
|---|---|---|---|---|
| Red to | 5.000 | 20.02 | .013 (45 degree) | 750 |
| Green/Yellow | | | | |
| (2^{nd} loop) | 5.000 | 20.02 | .013 (45 degree) | 750 |

### Example 4

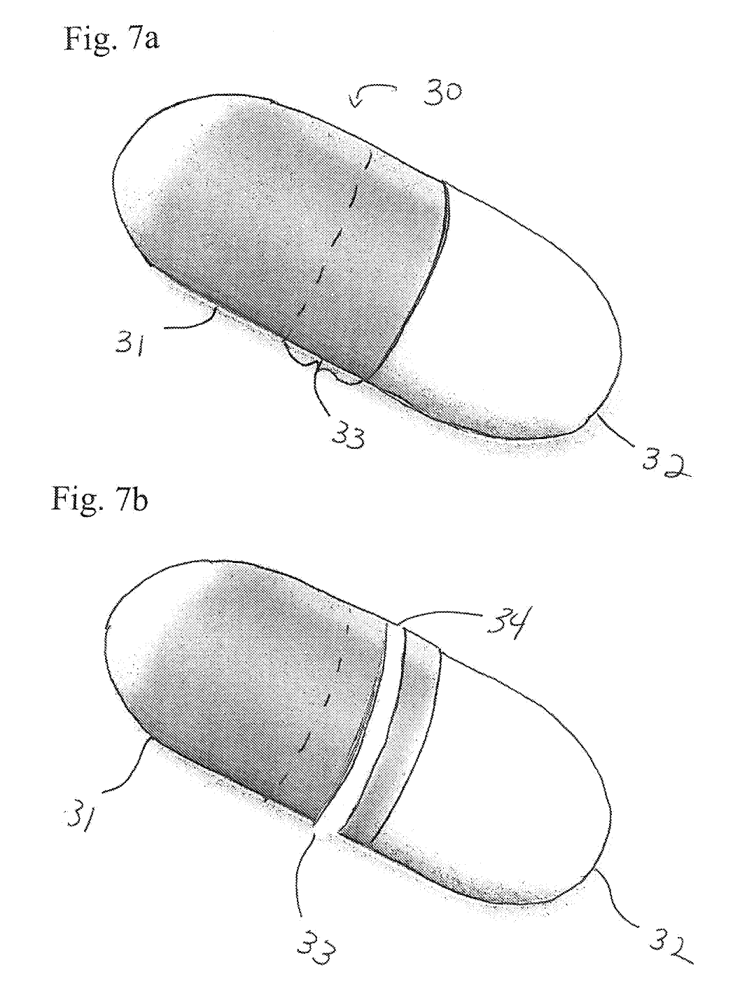

In this example, capsules containing acetaminophen are laser etched according to a process of the invention. Specifically, the capsules are prepared using standard pharmaceutical manufacturing techniques so that each capsule contains approximately 500 mg of the drug and the capsules used are specifically designed so that there is approximately a 20% overlap occurring after assembly. Referring now to FIGS. 7A and 7B, there is shown a capsule 30 having an upper portion 1 having a first color, such as green, while the lower portion of the capsule 32 has a second color, such as yellow. As a result of the overlap 33, the uppermost section of the yellow, lower portion 32 of the capsule is unseen. See FIG. 7A. A YAG laser is used to remove a single band 33 of the green capsule material and expose the yellow underneath to reveal a capsule having a unique appearance. See FIG. 7B.

Although the description above has generally focused on forming a pattern having a color gradient or variation on a pharmaceutical tablet, the method and arrangement in accordance with the invention can be used to form a pattern having a color gradient ton other pharmaceutical dosage forms.

Having described exemplary embodiments of the invention with reference to the accompanying drawings, it will be appreciated that the present invention is not limited to those embodiments, and that various changes and modifications can be effected therein by one of ordinary skill in the art without departing from the scope or spirit of the invention as defined by the appended claims. For example, it may be possible to apply a top coat over the coating layer after the laser etching process. Application of the top coat in this embodiment would provide the tablet with a substantially uniform appearance, i.e., fill in any minute depressions in the outermost coating resulting from the laser etching.

1. A method for forming pharmaceutical tablets having a laser etched region thereon optionally providing a color gradient thereon, comprising:
applying at least one color coating onto a tablet core to form a coating layer, the at least one color coating having a color different than the color of the tablet core; and
laser etching a portion of the coating layer to remove a selected portion thereof and render the color of the tablet core is visible at the location at which the selected portion of the coating layer has been removed.

2. (canceled)

3. The method of claim 1, wherein the selected portion of the coating layer being removed comprises individual pixels, further comprising adjusting the density and size of the pixels in a linear direction of the tablet core and wherein the laser is optionally controlled to form a variation in the thickness of the coating layer from one side of the pattern to an opposite side of the pattern.

4. The method of claim 1, wherein the selected portion of the coating layer removed comprises a non-pixelated region of the pharmaceutical tablet.

5. The method of claim 1, wherein the laser etching step comprises a multi-step laser etching process, further comprising controlling the laser such that different thicknesses of different portions of the coating layer are removed in each laser etching step.

6. (canceled)

7. The method of claim 1, wherein the laser etching step comprises providing a plurality of lasers, laser etching a first pattern onto the coating layer by means of a first one of the lasers and laser etching a second pattern different than the first pattern onto the coating layer by means of a second one of the lasers.

8. The method of claim 1, wherein the laser etching step comprises providing a single laser, laser etching a first pattern onto the coating layer by means of the single laser, changing the operating parameters of the laser and then laser etching a second pattern different than the first pattern onto the coating layer by means of the single laser.

9. The method of claim 1, wherein the laser etching step comprises orienting the laser at least one angle which is not perpendicular to a horizontal axis or surface of each tablet.

10. The method of claim 1, wherein the laser etching step is a multi-step laser etching process and comprises orienting the laser at a different angle which is not perpendicular to a horizontal axis or surface of each tablet in each laser etching step.

11. The method of claim 1, wherein the at least one coating comprises a plurality of coatings, the laser etching step is a multi-step laser etching process and comprises:
orienting the laser at a different angle which is not perpendicular to a horizontal axis or surface of each tablet in each laser etching step; and
controlling the laser such that different thicknesses of the coating layer are removed in each laser etching step.

12. The method of claim 1, wherein the at least one coating is applied onto the tablet core by a process selected from a group consisting of spraying, enrobing, gel dipping, sugar-coating and electrostatic coating.

13. (canceled)

14. The method of claim 1, wherein said at least one coating includes a first coating layer enveloping the entire tablet core, and a second coating layer covering the entire first coating layer, and optionally a third coating layer covering said second coating layer, each of said first, said second and said third coating layers being of different colors.

15. (canceled)

16. The method of claim 1, wherein said at least one coating includes said first, second and third coating layers, further comprising:
a) laser etching a first design onto the tablet core by removing contiguous portions of each of said first, said second and said third coating layers; and
b) laser etching a second design onto said first tablet layer of the pharmaceutical tablet, separate from said first design, by removing contiguous portions of each of said second and said third coating layers, and optionally further comprising:
c. laser etching a third design onto the pharmaceutical tablet, separate from each of the first and the second designs, by removing a portion of said third coating layers.

17. (canceled)

18. The method of claim 1, wherein a plurality of coatings are applied onto the tablet core, further comprising applying a first one of the coatings onto the entire tablet core, and applying a second coating onto only a portion of the first coating such that a portion of the first coating is not covered by the second coating, optionally further comprising selecting colors of the coatings and a color of the tablet core to provide a high contrast between them.

19. (canceled)

20. (canceled)

21. A pharmaceutical tablet prepared by the process of claim 1.

22. A pharmaceutical tablet having a laser etched region thereon prepared by forming a coating layer having a first color onto a tablet core having a second color, the first color being different than the second color; and laser etching a portion of the coating layer to remove a selected portion thereon to reveal the second color of the tablet core where the selected portion of the coating layer has been removed.

23. An arrangement for forming pharmaceutical tablets with a laser etched region or color gradient thereon, comprising:
at least one coating system arranged to apply at least one color coating onto tablet cores to thereby form a coating layer, the at least one color coating having a color different than the color of the tablet cores; and
a laser etching system arranged to remove a selected portion of said coating layer and thereby provide a laser etched region thereon or cause a variation in said coating layer whereby the color of the tablet cores is visible at locations at which the selected portions of said coating layer have been removed, and optionally further comprising:
an inspecting system arranged downstream of said laser etching system and arranged to inspect the laser etched tablets to verify correct formation of the color variation.

24. The arrangement of claim 23, wherein said laser etching system includes at least one laser, said at least one laser being controllable to adjust a density and size of the removed portions in a linear direction of the tablet cores and optionally being arranged to be oriented at least one angle which is not perpendicular to a horizontal axis or surface of each tablet core to thereby enable formation of a holographic pattern.

25. The arrangement of claim 23, wherein said laser etching system comprises a plurality of lasers which perform multiple laser etching steps on the same tablet cores, said lasers being controllable such that different thicknesses of different portions of said coating layer are removed in each laser etching step, said lasers being optionally controlled to form a variation in the thickness of said coating layer from one side of the pattern to an opposite side of the pattern.

26-29. (canceled)

30. The arrangement of claim 23, wherein said at least one coating system is arranged to apply a plurality of coatings onto the tablet cores, at least one of said coatings being applied onto only a portion of an outermost surface on the tablet cores.

31-40. (canceled)

41. A method for forming pharmaceutically acceptable capsule having a laser etched region thereon, comprising:
encapsulating a pharmaceutical dosage form, with a pharmaceutically acceptable capsule comprising an upper shell portion and a lower shell portion, which may be of different colors, said upper shell portion covering at a portion the lower shell portion upon the encapsulating of the pharmaceutical dosage form; and
laser etching a portion of the upper shell portion covering the lower shell portion to remove a selected portion thereof and render the lower shell portion of the capsule visible at the location at which the selected portion of the upper shell portion has been removed.

42. (canceled)

43. The method of claim 41, wherein the pharmaceutical dosage form is a compressed tablet which is optionally film coated with a color which is not the same as the color of the upper shell portion, the lower shell portion or both the upper and lower capsule shell portions.

44-45. (canceled)
